(19) United States
(12) Patent Application Publication
(10) Pub. No.: US 2024/0057986 A1
(43) Pub. Date: Feb. 22, 2024

(54) INDUCTIVELY COUPLED FOOT PEDAL BATTERY CHARGING

(71) Applicant: Alcon Inc., Fribourg (CH)

(72) Inventors: Steven T. Charles, Memphis, TN (US); Sean Christopher Madden, Mission Viejo, CA (US); Gary P. Sorensen, Mission Viejo, CA (US)

(21) Appl. No.: 18/449,776

(22) Filed: Aug. 15, 2023

Related U.S. Application Data
(60) Provisional application No. 63/371,563, filed on Aug. 16, 2022.

(51) Int. Cl. A61B 17/00; H02J 50/10; H02J 50/00

(52) U.S. Cl. CPC A61B 17/00; H02J 50/10; H02J 50/005; H02J 50/80

(57) ABSTRACT

A surgical system for wirelessly charging a foot controller is described. The surgical system includes a surgical console. The surgical system also includes a foot controller wirelessly coupled to the surgical console and adapted to control one or more operations of the surgical console. The surgical system further includes a charging apparatus coupled to the surgical console and adapted to wirelessly charge the foot controller.

### BACKGROUND

When surgically treating a patient, a surgeon generally uses a surgical system that requires the control of a variety of different pneumatic and electronically driven subsystems. Operation of the various subsystems is generally controlled by a processor driven console. The processor receives mechanical or electronic inputs from the surgeon or other medical professionals to control the operational characteristics of the various subsystems.

In ophthalmic surgical systems, foot controllers connected to the console are generally used to control a variety of surgical subsystems. To control the surgical console and its associated handpieces during the various stages of the surgical procedure, the surgeon may use a foot controller to perform a variety of operations (e.g., changing settings on a surgical console and activating, de-activating, or changing the operations of a hand-piece, probe, etc.), during a variety of ophthalmic surgical procedures, such as cataract and vitreo-retinal procedure.

Some ophthalmic surgical systems employ wireless foot controllers that are communicatively coupled to the surgical console. One challenge with wireless foot controllers is that they are powered via batteries, which have to be frequently charged in order for the wireless foot controllers to operate. The charging is typically done by physically connecting the wireless foot controller to the console with a charging cable. Charging wireless foot controllers in this manner has many deficiencies and can pose many challenges, such as a safety risk to medical professionals in the operating room, as described in more detail herein. Therefore, there is a need for an improved system for charging a wireless foot controller.

### SUMMARY

In certain embodiments, a surgical system is provided. The surgical system includes a surgical console. The surgical system also includes a foot controller wirelessly coupled to the surgical console and adapted to control one or more operations of the surgical console. The surgical system further includes a charging apparatus coupled to the surgical console and adapted to wirelessly charge the foot controller.

The following description and the related drawings set forth in detail certain illustrative features of one or more embodiments.

### BRIEF DESCRIPTION OF THE DRAWINGS

The appended figures depict certain aspects of one or more disclosed embodiments and are therefore not to be considered limiting of the scope of this disclosure.

FIG. 1 illustrates a perspective view of an example surgical system, according to certain embodiments.

FIG. 2 is a block diagram illustrating exemplary components of the surgical system of FIG. 1, according to certain embodiments.

FIG. 3 illustrates a schematic view of a charging apparatus sized to accommodate multiple foot controllers and/or multiple foot controller positions.

To facilitate understanding, identical reference numerals have been used, where possible, to designate identical elements that are common to the drawings. It is contemplated that elements and features of one embodiment may be beneficially incorporated in other embodiments without further recitation.

### DETAILED DESCRIPTION

Embodiments described herein provide systems for charging a wireless foot controller used to control a surgical system.

In conventional surgical systems that employ wireless foot controllers, the charging of the wireless foot controllers is typically performed via a cable connection to the surgical console. That is, the wireless foot controllers may be physically connected to the surgical console via a cable connector in order to supply power from the surgical console to the batteries of the wireless foot controller. However, because the wireless foot controller may not be operational while it is being charged, charging a wireless foot controller in this manner can reduce its usefulness in a surgical setting, as its operational time may be impacted by the battery life of the wireless foot controller. Additionally, in some instances, the physical cable connection between the wireless foot controller and the surgical console can pose safety risks (or safety hazards) to medical personnel.

Accordingly, embodiments described herein provide a surgical system that includes a wireless foot controller (also referred to herein as a wireless foot pedal) and a charging apparatus inductively coupled to the wireless foot controller. The charging apparatus is adapted to charge one or more batteries of the wireless foot controller via the inductive coupling. In certain embodiments, the charging apparatus is configured as a floor mat (also referred to herein as a foot pad) that provides support for the wireless foot controller. The floor mat may include an enclosed coil that is inductively coupled to a coil on the bottom surface of the wireless foot controller (overlaying the floor mat).

In addition to, or as an alternative to, the wireless foot controller being charged via the inductive coupling, in certain embodiments, the wireless foot controller may communicate (e.g., send and/or receive) data (e.g., control signals) using the inductive coupling. For example, the charging apparatus may be physically connected to the surgical console via a cable. In this example, the wireless foot controller may exchange communications with the charging apparatus via the inductive coupling, and the charging apparatus may be adapted to exchange the communications with the surgical console via the cable. In certain embodiments, the inductive coupling between the charging apparatus and the wireless foot controller can provide the only communication link through which communication between the wireless foot controller and the surgical console takes place. In certain other embodiments, the inductive coupling between the charging apparatus and the wireless foot controller can provide a secondary (redundant) communication link in situations where the primary (wireless) communication link between the wireless foot controller and surgical console encounters a link failure or is otherwise unavailable. Additionally or alternatively, the inductive coupling between the charging apparatus and the wireless foot controller may allow the surgical console to verify that the communications received via the primary communication link are accurate and/or reliable.

In certain embodiments, the cable between the charging apparatus and the surgical console may have a form factor that reduces the likelihood of the cable presenting a safety risk (or safety hazard) to medical personnel. For example, in certain embodiments, the cable is a flat ribbon cable, instead of a conventional cable with a circular cross section typically used in conventional surgical systems.

By providing a system for wirelessly charging a wireless foot controller, embodiments may allow for continuously charging the wireless foot controller increasing the operational time of the wireless foot controller. Additionally, continuous charging of the wireless foot controller may enable the wireless foot controller to use a high torque, high current motor for fluidics resistance feedback.

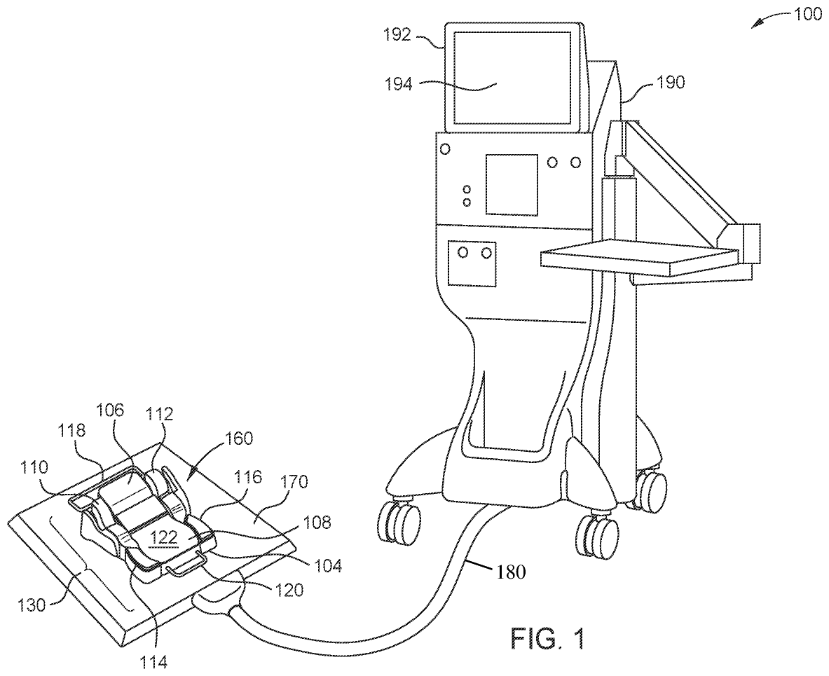

FIG. 1 illustrates a perspective view of an example surgical system 100, according to certain embodiments. The surgical system 100 includes a surgical console 190 (also referred to herein more generally as a console), a charging apparatus 170, and a foot controller 160. The surgical console 190 may be operably coupled, physically and/or wirelessly, to any number of user interfaces and/or devices. Here, for example, the surgical console 190 is operably coupled, physically, to the charging apparatus 170 via a cable 180, and is operably coupled, wirelessly, to the foot controller 160.

The foot controller 160 includes a body 130 with a base 104. The base 104 may support the foot controller 160 on the operating room floor or on the charging apparatus 170 disposed on the operating room floor. The body 130 includes a footpedal 106, a heel rest 108, a left toe switch 110, a right toe switch 112, a left heel switch 114, and a right heel switch 116. A first handle 118 and a second handle 120 are coupled to the body 130. Note that the configuration of switches, handles, and footpedals of the foot controller 160 depicted in FIG. 1 are provided as reference examples. It is contemplated that the foot controller 160 may have any suitable number and configuration of switches, handles, and footpedals, which are configured to be actuated by a user in a defined sequence, e.g., to enter a password, perform one or more actions of a surgical procedure, etc.

A surgeon can use footpedal 106 for proportional control of certain functions or surgical parameters during a surgical procedure. For example, the surgeon can depress the footpedal 106 using the upper portion of the surgeon's foot to move from a fully undepressed to, for example, a fully depressed position in which the footpedal 106 lies in generally the same plane as the heel rest 108. The left toe switch 110 and the right toe switch 112 are generally dual mode binary switches that can be vertically or horizontally actuated to control certain functions or surgical parameters. For example, a first mode may be actuated when a surgeon presses downward on the left toe switch 110 or the right toe switch 112. A second mode may be actuated when the surgeon presses in a generally outward, horizontal direction on the left toe switch 110 or the right toe switch 112 with the side of his or her foot. The left heel switch 114 and right heel switch 116 are generally binary switches that are actuated when a surgeon presses downward with his or her heel.

The surgical console 190 allows a user, generally a surgeon or other medical personnel, to begin a surgical procedure by setting the initial operating parameters and modes into the surgical console 190, for example by using an electronic display screen 192 (e.g., via a touch-screen interface, mouse, trackball, keyboard, etc.), which includes a graphical user interface (GUI) 194. The electronic display screen 192 allows the user to access various menus and screens related to the functions and operations of the surgical console 190. The electronic display screen 192 may be controlled by a processor coupled to a memory (e.g., random access memory (RAM)). The instructions stored in the memory configure the processor to execute one or more operations, such as displaying the various menus and screens on electronic display screen 192 as well as other operations described herein. For example, as the user advances through the surgical procedure, user input regarding changes to the operating modes and parameters can be received by the processor, which executes instructions stored in memory based on that input and controls the electronic display screen 192. In this example, at least some of the user input may be received from the foot controller 160.

One or more users, generally a surgeon or another medical professional, interacts with the graphical user interface 194 throughout the various stages of the surgical procedure. For example, the user, or another medical professional in the operating room, may toggle from one stage of the procedure to the next by selecting the next stage on the graphical user interface 194. The user may also toggle to the next stage using one or more of the left toe switch 110, the right toe switch 112, the left heel switch 114, or the right heel switch 116 of the foot controller 160. As an example, during certain surgical procedures, such as vitrectomy, one such stage is a laser photocoagulation stage (“laser stage”) during which a laser is used to treat the patient, for example to reattach the retina of the patient by cauterizing it together with the inner surface of the uvea using a laser beam. Only upon entering this stage is it possible to enable laser emission control, by pressing or otherwise actuating toe switches in a defined sequence. Other stages of surgical procedures, such as a vitrectomy, include, for example, a ready state and a laser emission state, which may be entered (or activated) by a user via the foot controller 160. Thus, the foot controller 160 serves as an integrated foot controller that allows for the switches and pedal to be used to step through the various stages of a surgical procedure and to be used to control the operations of surgical console 190 as well as various handheld surgical devices, such as a laser probe used for photocoagulation, an illumination probe, a vitrectomy probe, etc.

As shown in FIG. 1, the foot controller 160 may also include one or more sensors 122. A single sensor 122 disposed on the heel rest 108 is shown as an example. The one or more sensors 122 are generally any sensor capable of collecting data to indicate whether the user's foot is on, or within a predetermined distance from, the footpedal 106. Suitable sensors include, but are not limited to, photosensors or photodiodes positioned to reliably sense the presence of a human foot. In some examples, the one or more sensors 122 include a pair of photosensors (a first photosensor and a second photosensor) or a pair of photodiodes (a first photodiode and a second photodiode) positioned to create a beam that is interrupted when the user's foot is present. For example, the first photosensor or photodiode may be coupled to the left side of the foot controller 100 and the second photosensor or photodiode may be coupled to the right side of the foot controller 100, such as the first photodetector being coupled to the left toe switch 110 and the second photodetector being coupled to the right toe switch 112. In another example, the one or more sensors 122 are reflective-type photodetectors located in the surface of the foot controller 100 where the user places his or her heel, such as in the heel rest 108. In yet another example, the one or more sensors 122 are two or more transmission photodetectors located on features on each side of the user's foot, such as in the left toe switch 110, the right toe switch 112, the left heel switch 114, or the right heel switch 116. For example, the first photodetector may be coupled to the left toe switch 110 and the second photodetector may be coupled to the right toe switch 112.

As noted, the surgical console 190 is operably coupled, wirelessly, to the foot controller 160. That is, the foot controller 160 may communicate control signals (responsive to the user using the various switches, sensors, and/or pedal(s) of the foot controller 160) to the surgical console using a wireless communication protocol (e.g., cellular communication protocol, 802.11, Bluetooth, etc.). In conventional surgical systems that employ wireless foot controllers, such as the foot controller 160, the charging of the wireless foot controllers generally involves physically connecting the wireless foot controller to a power source with a cable in order to charge the batteries of the wireless foot controller. As noted, however, charging wireless foot controllers in this manner is not ideal since it can reduce the amount of time that the foot controller is in operation, present safety hazards to medical personnel, etc.

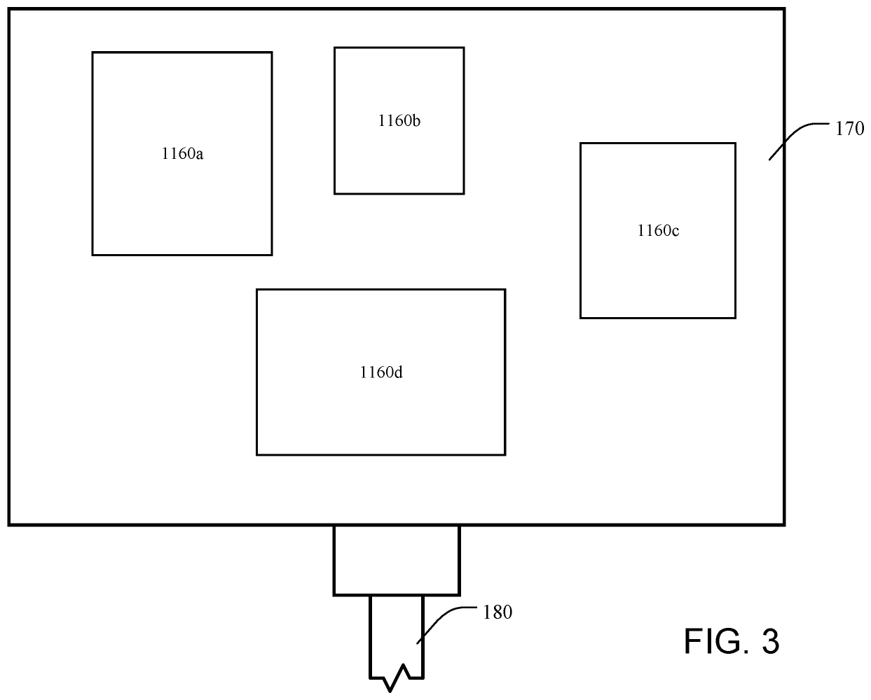

As such, the surgical system 100 depicted in FIG. 1 utilizes a charging apparatus 170 to charge the foot controller 160, via inductive coupling. The charging apparatus 170 is generally a wireless power transmitter that is configured to wirelessly transmit power to one or more batteries of the foot controller 160. Similarly, the foot controller 160 is generally a wireless power receiver that is configured to wirelessly receive power from the charging apparatus 170. As described below, the charging apparatus 170 includes a coil that inductively couples to a coil in the foot controller 160, when, for example, the foot controller 160 is disposed on (or placed on) the charging apparatus 170. In the embodiment depicted in FIG. 1, the charging apparatus 170 is in the form of a floor mat (also referred to as a foot pad). However, note that the charging apparatus 170 can have any suitable form factor consistent with the functionality described herein. For example, while the charging apparatus 170 in FIG. 1 is shown large enough to accommodate a single foot controller 160, a larger charging apparatus 170 is shown in FIG. 3. As seen in FIG. 3, the charging apparatus may be large enough to accommodate multiple foot controllers 1160a-b of different shapes and sizes. For example, a user may have both a foot controller for phacoemulsification control and a separate foot controller for laser control on the charging apparatus 170 at the same time. Furthermore, a larger charging apparatus 170 may accommodate multiple positions for a foot controller 1160 (or multiple foot controllers). For example, as seen in FIG. 3, the charging apparatus 170 may be large enough to accommodate at least a first and a second position for the foot controller that do not overlap. This may make it easier for a user to move the foot controller 1160 to a comfortable position or re-position the foot controller 1160 as needed during a surgery while still maintaining contact between the foot controller 1160 and the charging apparatus 170. The charging apparatus 170 can be formed from a variety of materials, including, for example, rubber, plastic, etc.

As shown in FIG. 1, the charging apparatus 170 is physically coupled to the surgical console 190 via a cable 180. The cable 180 is generally configured to provide power to the charging apparatus 170. In certain embodiments, the cable 180 also provides a physical communications link between the charging apparatus 170 and the surgical console 190. For example, the charging apparatus 170 may receive data from the foot controller 160 via the inductive coupling between the charging apparatus 170 and the foot controller 160, and may send the data to the surgical console 190 via the cable 180. By using the inductive coupling and cable 180 to transmit data to the surgical console 190, embodiments provide a redundant communication link for the surgical console 190 to receive control signals from the foot controller 160. Additionally, the redundant communication link may enable the surgical console 190 to verify that control signals received via a primary wireless communication link from the foot controller 160 are accurate.

The cable 180 may have a form factor that reduces the likelihood of the cable 180 presenting a safety risk (or safety hazard). For example, as opposed to having a circular cross section, the cable 180 may be a flat ribbon cable. Additionally, in certain embodiments, the cable 180 may be coupled to the charging apparatus 170 via an overmolding to provide the cable 180 improved protection from fluids, shock, vibration, flexing, etc. In these embodiments, the cable 180 may be referred to as an overmolded cable (or overmolded cable assembly).

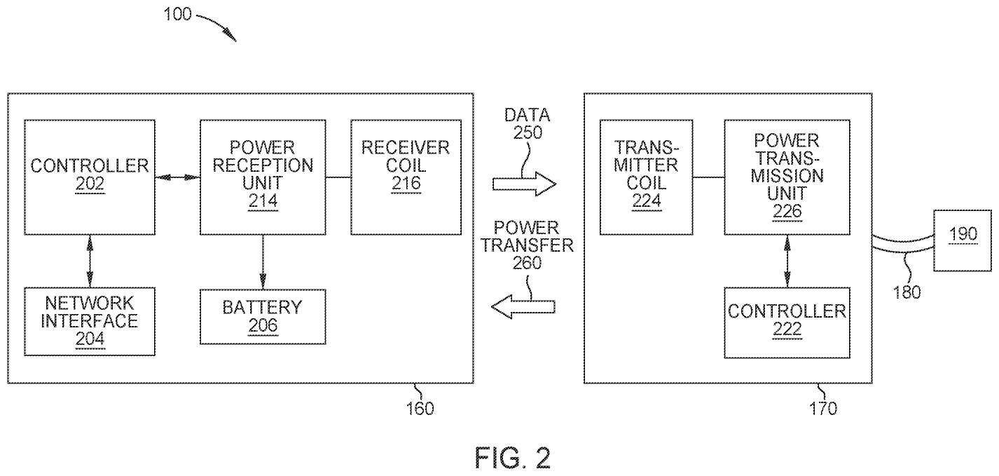

FIG. 2 is a block diagram illustrating components of the surgical system 100, described relative to FIG. 1, according to certain embodiments. Note FIG. 2 illustrates a reference example of how various components of the surgical system 100 can communicate and operate together, and is not intended as the sole implementation of the surgical system 100.

As shown, the charging apparatus 170 of the surgical system 100 includes, without limitation, a transmitter coil 224, a power transmission unit 226, and a controller 222. The power transmission unit 226 may wirelessly supply power (e.g., power transfer 260) to the power reception unit 214 of the foot controller 160, via an inductive coupling between the transmitter coil 224 of the charging apparatus 170 and the receiver coil 216 of the foot controller 160. The power transmission unit 226 may supply power in an alternating current (AC) waveform or a direct current (DC) waveform. The power transmission unit 226 may be provided in the form of a built-in battery or may be provided in the form of a power receiving interface that receives power from an external component (e.g., surgical console 190) to supply power to another component (e.g., foot controller 160).

The controller 222 generally controls overall operations of the charging apparatus 170. For example, the controller 222 can control operations of the power transmission unit 226 using an algorithm, a program, or an application. The controller 222 may be provided as a central processing unit (CPU), a microprocessor, or a minicomputer. In certain embodiments, the controller 222 may send a charging control signal to the power transmission unit 226 to control one or more parameters of the wireless power transmitted from the power transmission unit 226.

Additionally, as shown, the foot controller 160 includes, without limitation, a controller 202, a network interface 204, a battery 206, a power reception unit 214, and a receiver coil 216. The power reception unit 214 may wirelessly receive power (e.g., power transfer 260) transmitted from the power transmission unit 226 via the inductive coupling between the receiver coil 216 and the transmitter coil 224. The power reception unit 214 may receive power in an AC waveform or DC waveform.

The controller 202 generally controls overall operations of the foot controller 160. For example, the controller 202 can control operations of the network interface 204, power reception unit 214, etc. The controller 202 may be provided as a CPU, a microprocessor, or a minicomputer. In certain embodiments, the controller 222 may send a charging control signal to the power reception unit 214 to control a charging function of the battery 206. For example, the controller 222 may control, via the power reception unit 214, the amount of power that is supplied from the power reception unit 214 to the battery 206.

The network interface 204 is generally configured to communicate with one or more devices, including, for example, surgical console 190, using a wireless communication protocol. The wireless communication protocol can be any suitable wireless communication protocol, including, for example, 802.11, a cellular communication protocol (e.g., 5G, 4G, 3G, etc.), Bluetooth, ZigBee, etc. In certain embodiments, the foot controller 160 may establish a primary communication link with the surgical console 190 via the network interface 204.

In certain embodiments, the foot controller 160 may also establish a secondary communication link with the surgical console 190 via the inductive coupling and cable 180. For example, as shown, the foot controller 160 may send data 250 via the inductive coupling to the charging apparatus 170, which may send (or forward) the data 250 to the surgical console 190.

The foregoing description is provided to enable any person skilled in the art to practice the various embodiments described herein. Various modifications to these embodiments will be readily apparent to those skilled in the art, and the generic principles defined herein may be applied to other embodiments. Thus, the claims are not intended to be limited to the embodiments shown herein, but are to be accorded the full scope consistent with the language of the claims.

What is claimed is:

1. A surgical system comprising:
a surgical console;
a foot controller wirelessly coupled to the surgical console and adapted to control one or more operations of the surgical console; and
a charging apparatus coupled to the surgical console and adapted to wirelessly charge the foot controller.

2. The surgical system of claim 1, wherein the charging apparatus is coupled to the surgical console via a cable.

3. The surgical system of claim 2, wherein the cable is a flat ribbon cable.

4. The surgical system of claim 2, wherein the charging apparatus is coupled to the cable via an overmolding.

5. The surgical system of claim 1, wherein:
the charging apparatus comprises a first coil; and
the foot controller comprises a second coil.

6. The surgical system of claim 5, wherein the charging apparatus is adapted to wirelessly transmit power to the foot controller via an inductive coupling between the first coil and the second coil.

7. The surgical system of claim 6, wherein the inductive coupling is established when the foot controller is overlaid on a top surface of the charging apparatus.

8. The surgical system of claim 5, wherein:
the foot controller is adapted to transmit data to the charging apparatus via an inductive coupling between the first coil and the second coil; and
the charging apparatus is configured to forward the data to the surgical console via a cable coupling the charging apparatus to the surgical console.

9. The surgical system of claim 1, wherein the charging apparatus is a floor mat.

10. The surgical system of claim 9, wherein the floor mat is comprised of a rubber material.

11. The surgical system of claim 1, wherein the one or more operations comprise putting the surgical console in one or more states of a surgical procedure.

12. The surgical system of claim 1, wherein the charging apparatus is sized to accommodate the foot controller and at least one additional foot controller.

13. The surgical system of claim 1, wherein the charging apparatus is sized to accommodate at least a first and a second position for the foot controller that do not overlap.
